(19) United States
(12) Patent Application Publication
(10) Pub. No.: US 2026/0020991 A1
(43) Pub. Date: Jan. 22, 2026

(54) PANT-TYPE ABSORBENT ARTICLE

(71) Applicant: The Procter & Gamble Company, Cincinnati, OH (US)

(72) Inventors: Fengchun YE, Beijing (CN); Rong FU, Beijing (CN); Na WU, Beijing (CN); Meng CHEN, Beijing (CN); Hui LIU, Beijing (CN)

(21) Appl. No.: 19/337,060

(22) Filed: Sep. 23, 2025

Related U.S. Application Data
(63) Continuation of application No. PCT/CN2024/072950, filed on Jan. 18, 2024.

(30) Foreign Application Priority Data
Apr. 13, 2023 (WO) PCT/CN2023/088116

(51) Int. Cl. A61F 13/496; A61F 13/15; A61F 13/49; A61F 13/494

(52) U.S. Cl. CPC A61F 13/496; A61F 13/49011; A61F 13/49466; A61F 2013/15552

(57) ABSTRACT

Disclosed is an absorbent article comprising: a front elastic belt transversely extending between the pair of side seams; a back elastic belt transversely extending between the pair of side seams; a crotch region longitudinally extending between the front and back elastic belts; and an absorbent main body extending the entire longitudinal dimension of the crotch region and further extending partly into each of the front elastic belt and the back elastic belt. A waist guard portion extends towards the crotch region from a transversely extending closed end line towards an open edge.

### CROSS REFERENCE TO RELATED APPLICATION

This application is a continuation, under 35 USC 120, of PCT Application No. PCT/CN2024/072950, filed on Jan. 18, 2024, which claims priority to PCT Application No. PCT/CN2023/088116, filed on Apr. 13, 2023, which are both herein incorporated by reference.

### FIELD

The present invention relates to pant-type absorbent articles having a protective measure for preventing leakage of low viscosity bodily exudates, while also maintaining other functions as a pant-type wearable article.

### BACKGROUND

Infants and other individuals wear absorbent articles such as diapers to receive and contain urine and other body exudates. Pull-on absorbent articles, or pant-type absorbent articles, are those which are donned by inserting the wearer's legs into the leg openings and sliding the article up into position about the lower torso. Pant-type absorbent articles having a protective measure for preventing leakage of low viscosity bodily exudates through the waist opening are known, for example, in WO2021/169166A.

Pant-type articles may take various structures wherein the circumference of the waist opening and vicinity thereof is made elastic enough to facilitate the wearer or the caregiver to expand the article and insert the wearer's legs into the leg openings for wearing the article. Accordingly, pant-type articles provide only a very small range of size adjustment or body configuration adjustment based on the structural limitations of the article. As such, pant-type articles are typically so configured to accommodate size and configuration ranges by providing the elastic belt region very stretchable and comfortable to wear, yet with reliable fit such that sufficient protection against sagging and urine leakage may be provided. It is desired that such basic functions of the elastic belt region may be maintained even with introduction of the aforementioned protective measure.

Based on the foregoing, there is a need for an absorbent article provided with a protective measure for preventing leakage of low viscosity bodily exudates from the waist opening, while maintaining good fit, comfort during wear, urine leakage prevention, and softness. There is also a need for providing such an absorbent article which can be economically made.

### SUMMARY

The present invention is directed to an absorbent article having a longitudinal direction, a transverse direction, a pair of side seams, and a pair of leg openings, comprising:
a front elastic belt transversely extending between the pair of side seams;
a back elastic belt transversely extending between the pair of side seams;
a crotch region longitudinally extending between the front and back elastic belts; and
an absorbent main body extending the entire longitudinal dimension of the crotch region and further extending partly into each of the front elastic belt and the back elastic belt, the absorbent main body comprising a liquid pervious topsheet, a liquid impervious backsheet, and an absorbent material region sandwiched therebetween, the absorbent main body having a maximum transverse dimension W2 which is from about 22% to about 50% of a transverse dimension of the back elastic belt W1;
the back elastic belt divided into a back waist zone, a protective zone, and a buttock zone lined up from the distal edge to the proximal edge, each zone spanning in the transverse direction and defined by its location from the distal edge to the proximal edge relative to the percentage of a longitudinal dimension LS of the side seam, wherein elastic members are disposed on each of the back waist zone, the protective zone, and the buttock zone,
the protective zone comprising a main portion and a waist guard portion superposed with each other, the waist guard portion extending towards the crotch region from a transversely extending closed end line towards an open edge, wherein the waist guard portion is partially bonded to the main portion to define a pair of closed side lines, wherein a pocket is formed by the closed end line, the pair of closed side lines, and the open edge, the pocket having an area A2 of from about 5% to about 15% of the back elastic belt having an area A1; and the closed end line is positioned proximal from the back distal edge of the backsheet;
wherein the waist guard portion comprises a waist guard clastic portion, wherein the main portion superposing the waist guard elastic portion is defined a correlated portion.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1 is a perspective view of one embodiment of an absorbent article of the present invention.

FIG. 2A is a schematic plan view of one embodiment of an absorbent article of the present invention with the side seams removed and in a flat uncontracted condition showing the garment facing surface.

FIG. 2B is a schematic plan view of another embodiment of an absorbent article of the present invention with the side seams removed and in a flat uncontracted condition showing the body-facing surface.

FIG. 3A-3C are schematic plan views of the back elastic belt of FIG. 2A with the seams unjoined and in a flat uncontracted condition showing the body-facing surface.

FIG. 4 is a schematic cross section view of the embodiment of FIGS. 2A and 3A-3C, taken along line L2, with the thickness dimension exaggerated.

FIG. 5 is a schematic view of an example of a hanger-type sample holding fixture according to the “Whole Article Force Measurement” herein.

FIG. 6A-6C are schematic views of an example of demonstration of vapor permeability and shape memory objects useful for such demonstration.

### DEFINITIONS

As used herein, the following terms shall have the meaning specified thereafter:

“Absorbent article” refers to articles of wear which may be in the form of pants, taped diapers, incontinent briefs, feminine hygiene garments, and the like, so configured to also absorb and contain various exudates such as urine, feces, and menses discharged from the body. The “absorbent article” may serve as an outer cover adaptable to be joined with a separable disposable absorbent insert for providing absorbent and containment function, such as those disclosed in PCT publication WO 2011/087503A.

“Pant” refers to disposable absorbent articles having a pre-formed waist and leg openings. A pant may be donned by inserting a wearer's legs into the leg openings and sliding the pant into position about the wearer's lower torso. Pants are also commonly referred to as “closed diapers”, “prefastened diapers”, “pull-on diapers”, “training pants” and “diaper-pants”.

“Longitudinal” refers to a direction running substantially perpendicular from a waist edge to an opposing waist edge of the article and generally parallel to the maximum linear dimension of the article.

“Transverse” refers to a direction perpendicular to the longitudinal direction.

“Proximal” and “distal” refer respectively to the position closer or farther relative to the longitudinal center of the article.

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate member(s) which in turn are affixed to the other element.

“Film” refers to a sheet-like material wherein the length and width of the material far exceed the thickness of the material. Typically, films have a thickness of about 0.5 mm or less.

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Extendibility” and “extensible” mean that the width or length of the component in a relaxed state can be extended or increased.

“Elasticated” and “elasticized” mean that a component comprises at least a portion made of clastic material.

“Elongatable material”, “extensible material”, or “stretchable material” are used interchangeably and refer to a material that, upon application of a biasing force, can stretch to an elongated length of at least about 110% of its relaxed, original length (i.e., can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Dimension”, “Length”, “Width”, “Pitch”, “Diameter”, “Aspect Ratio”, “Angle”, and “Area” of the article are all measured in a state wherein the article is extended to the Full Stretch Circumference W1 according to the “Whole Article Force Measurement” herein, and utilizing a ruler or a loupe, unless specified otherwise.

“Artwork” refers to a visual presentation to the naked eye, which is provided by printing or otherwise, and having a color. Printing includes various methods and apparatus well known to those skilled in the art such as lithographic, screen printing, flexographic, and gravure ink jet printing techniques.

“Color” or “Colored” as referred to herein includes any primary color except color white, i.e., black, red, blue, violet, orange, yellow, green, and indigo as well as any declination thereof or mixture thereof. The color white is defined as those colors having a L* value of at least 94, an a* value equal to 0±2, and a b* value equal to 0±2 according to the CIE L* a* b* color system.

### DETAILED DESCRIPTION

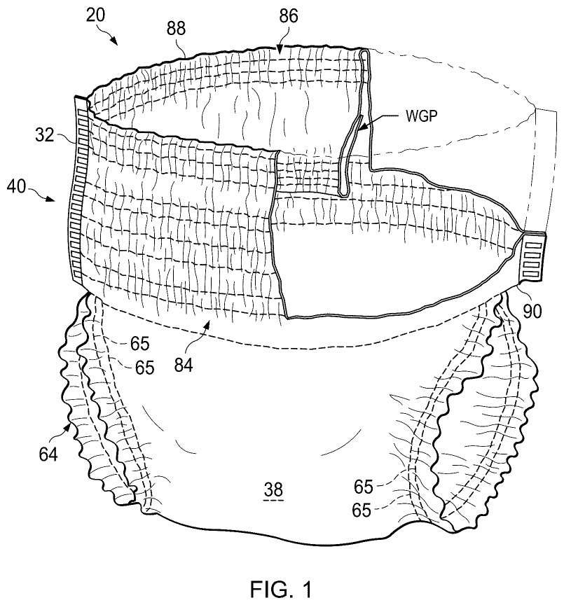

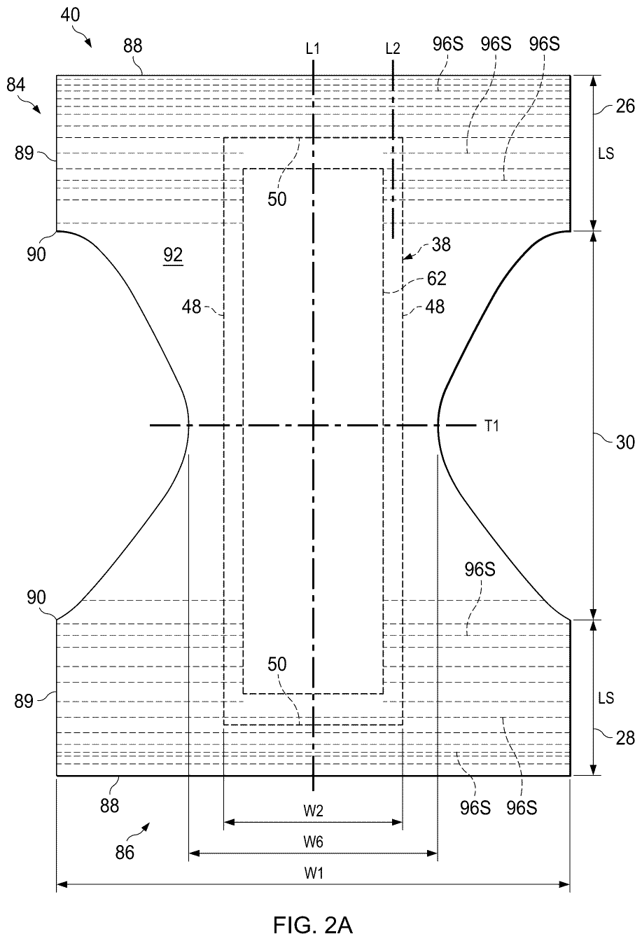

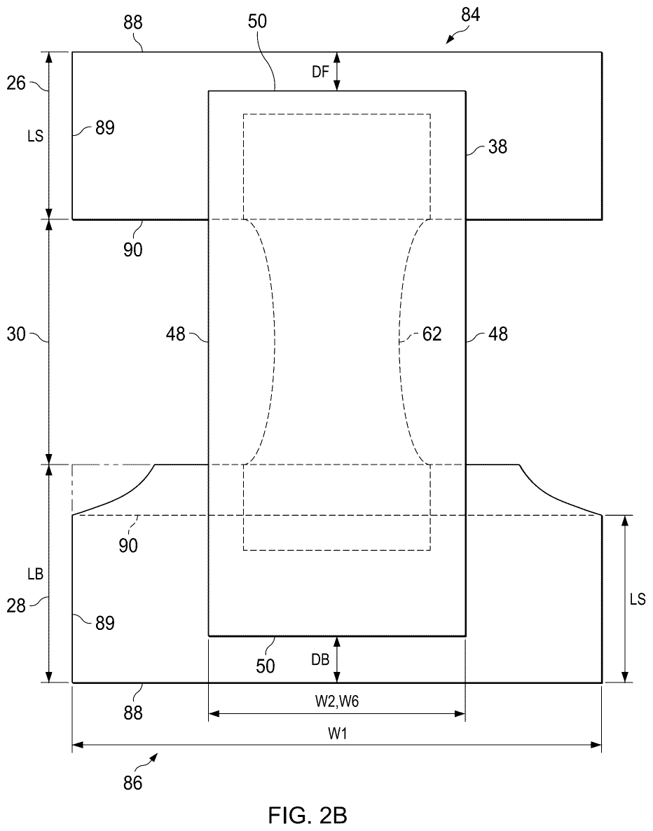

FIG. 1 is a perspective view of an absorbent article (20) of the present invention, and FIG. 2A is a schematic plan view of an absorbent article with the side seams (32) unjoined and removed, and in its flat uncontracted condition showing the garment-facing surface. The absorbent article (20) has a longitudinal centerline L1 which also serves as the longitudinal axis, and a transverse centerline T1 which also serves as the transverse axis. The absorbent article (20) has a body-facing surface, a garment facing surface, a front elastic belt (84), a back elastic belt (86), a crotch region (30), and side seams (32) which join the front elastic belt (84) and the back clastic belt (86), to form two leg openings and a waist opening. In FIG. 2A, the side edges (89) of the front and back elastic belt (84, 86) indicate the inward edges of the side seams (32).

The absorbent article (20) may be a uni-body type pant as in FIGS. 1 and 2A configured such that the garment facing layer of the absorbent main body (38) and the garment facing layer of the front and back elastic belts (84, 86) are common. For the uni-body type pant, the portion extending in the transverse direction between longitudinally extending side seams (32), respectively, are the front region (26), and the back region (28), and the remainder portion having curvilinear side edges is the crotch region (30), wherein the front elastic belt (84) matches the front region (26), and the back elastic belt (86) matches the back region (28). The absorbent article (20) may be a belt-type pant as in FIG. 2B formed by 3 separate parts forming the front region (26), the back region (28), and the absorbent main body (38). For the belt-type pant, the front elastic belt (84) and the back elastic belt (86) are portions extending in the transverse direction between the longitudinally extending side seams (32), thus the appendix belt existing proximal from the side seam (32) is not considered the back elastic belt (86). For either the uni-body type pant or the belt-type pant, the distal edge of the side seam (32) is considered the distal edge (88), and the proximal edge of the side seam (32) is considered the proximal edge (90). The distal edge (88) matches the waist opening.

The absorbent main body (38) comprises a topsheet, a backsheet and an absorbent material region (62) disposed between the topsheet and the backsheet. The topsheet may be a water permeable substrate. The backsheet may be a water impermeable film. The absorbent main body (38) comprises an absorbent material region (62) for absorbing and containing body exudates disposed on the absorbent main body (38), and an absorbent material non-existing region (61) surrounding the periphery of the absorbent material region (62). The area adjacent the front and back longitudinal end edges of the absorbent main body (38) comprise the backsheet. The absorbent material non-existing region (61) may be made only of the topsheet and the backsheet, and optional cuff materials as explained below. As shown in the embodiments of FIGS. 2A and 2B, the absorbent main body (38) may have a generally rectangular shape, left and right longitudinally extending side edges (48) and front and back transversely extending end edges (50). As shown in FIG. 2B, the absorbent material region (62) may have a generally hour glass shape. The absorbent material region (62) may exist through the entire longitudinal dimension of the crotch region and extending at least partly in the front and back regions (26, 28). The backsheet may extend the entire area of the absorbent main body (38). The absorbent main body has a maximum transverse dimension W2 which is from about 22% to about 50% of a transverse dimension of the back elastic belt W1. For the belt-type pant, the absorbent main body (38) may further comprise an outer cover layer (not shown) for covering the garment-facing side of the backsheet. The outer cover layer may be a nonwoven sheet.

Referring to FIGS. 1 and 2A, one preferred embodiment of the pant-type article of the present invention is the uni-body type which comprises an elastic cover (40) extending the entire longitudinal dimension of the article, and the absorbent main body (38), wherein the elastic cover (40) comprises the front elastic belt (84) and the back elastic belt (86). The clastic cover (40) may comprise a shaped crotch region, the shaped crotch region forming the leg openings. The clastic cover (40) may have a minimum transverse dimension W6 in the crotch region. By providing such shaped crotch region, the clastic cover (40) provides good fit and comfortable wear around the leg opening. Further, by providing the shaped crotch region to form the leg openings, the absorbent main body (38) may be provided in smaller transverse dimension than the clastic cover (40), also contributing to comfortable wear. Further, by providing the absorbent main body (38) in smaller transverse dimension, there is provided closer fit of the absorbent main body (38) to the wearer, particularly closer fit of the absorbent material existing region (62) to the wearer, that good handling of liquid exudates is effected. The minimum transverse dimension W6 of the clastic cover (40) may be from about 110% to about 150% of the maximum transverse dimension W2 of the absorbent main body (38). Referring to FIGS. 2A and 2B, the absorbent main body (38) may be rectangular in shape, namely having a consistent transverse dimension W2 along its entire length. The shaped crotch region of the elastic cover (40) may further comprise curved leg elastics (65) for forming leg opening cuffs (64) along the leg openings, as in FIG. 1.

The absorbent material region (62) may comprise an absorbent layer and an acquisition layer. The absorbent layer is the region wherein absorbent materials having a high retention capacity, such as superabsorbent polymers, are present. The absorbent layer may be substantially cellulose free. Superabsorbent polymers of the absorbent layer may be disposed between first and second layers of material immobilized by a fibrous layer of thermoplastic adhesive material. The first and second layers of materials may be nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. The acquisition layer facilitates the acquisition and the distribution of body exudates and may be placed between the topsheet and the absorbent layer. The acquisition layer may include cellulosic fibers.

The absorbent layers may be disposed in plurality in the absorbent material region (62). Some portions of the absorbent layers may be configured to have substantially no absorbent material to form a channel or a plurality of channels. Channels may be useful for allowing the absorbent material region (62) to bend upon swelling with fluids, such that the absorbent article conforms to the wearer's body after swelling and prevent sagging of the article. The channels may also be formed in the acquisition layer, and may be configured to at least partly match the channels of the absorbent layer in the thickness direction.

Alternatively, the absorbent material region (62) may comprise a high loft material encompassing superabsorbent polymers. The term “high loft” refers to low density bulky fabrics, as compared to flat, paper-like fabrics. High loft webs are characterized by a relatively high porosity. This means that there is a relatively high amount of void space in which superabsorbent polymer particles can be distributed. The high loft material (without the superabsorbent particles) may have a density at a pressure of 4.14 kPa (0.6 psi) below 0.20 g/cm³, in particular ranging from 0.05 g/cm³ to 0.15 g/cm³. The high loft layer (without the superabsorbent particles) may have a density at a pressure of 2.07 kPa (0.3 psi) below 0.20 g/cm³, in particular ranging from 0.02 g/cm³ to 0.15 g/cm³. The high loft layer (without the superabsorbent particles) may have a density at a pressure of 0.83 kPa (0.12 psi) below 0.15 g/cm³, in particular ranging from 0.01 g/cm³ to 0.15 g/cm³, and a basis weight of from 15 to 500 gsm, preferably 30˜200 gsm, such as those described in US 2021/0361497 A1. The absorbent material region (62) comprising high loft material encompassing superabsorbent polymers may also contain channels.

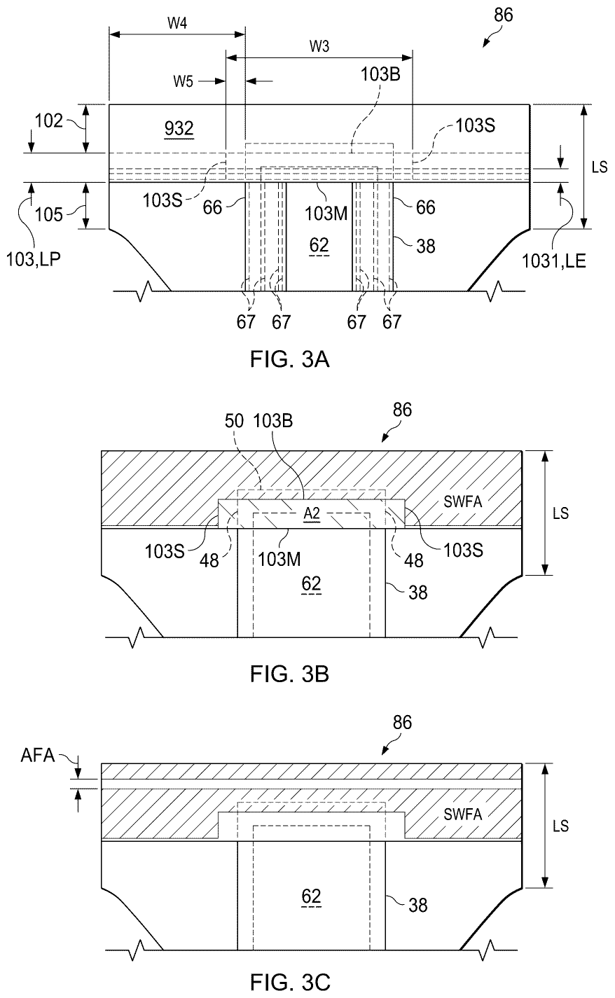

Referring to FIG. 1, the elastic cover (40) may comprise curved leg elastics for forming leg opening cuffs (64) along the leg openings, the outer cuffs being the same material forming the clastic cover (40). In addition, as in FIG. 3A, the absorbent main body (38) may comprise inner leg cuffs (66) made of material having high fluid impermeability, and elasticized to provide a barrier along both transverse edges of the absorbent main body (38). Such inner leg cuffs (66) may be attached to the remainder of the absorbent main body by bonding them, for example, to the topsheet at both longitudinal edges of the absorbent main body (38), such that at least in the crotch region (30), the inner leg cuffs (66) are in active elasticity to provide gasketing around the leg openings.

The front and back elastic belts (84, 86) of the article of the present invention acts to dynamically create fitment forces and to distribute the forces dynamically generated during wear. The front and back elastic belts (84, 86) are joined with each other outside the side edges (89) to form side seams (32), a waist opening and two leg openings. Each leg opening may be provided with elasticity around the perimeter of the leg opening. The elasticity around the leg opening may be provided by the leg opening elastics (65) provided along the shaped crotch region of the elastic cover (40) as in FIGS. 1 and 2A, or by the combination of elasticity from the front elastic belt (84), the back clastic belt (86), and the leg cuff elastics of the absorbent main body (38) as in FIG. 2B.

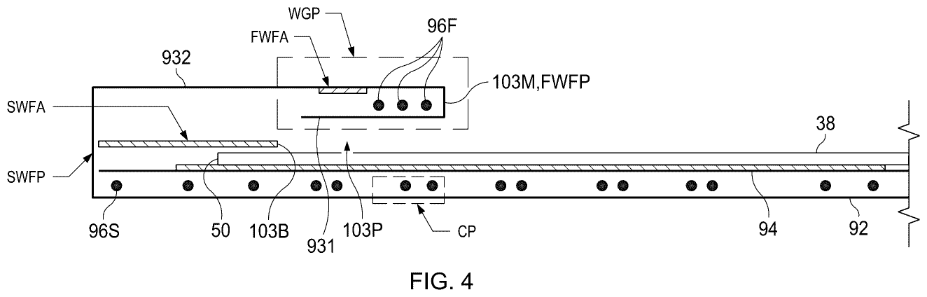

The front elastic belt (84) and back clastic belt (86) are configured to impart elasticity in the transverse direction. Referring to FIGS. 2A and 4, the front elastic belt (84) and the back elastic belt (86) may each comprise a laminate, the laminate comprising a plurality of elastic members (96F, 96S) running in the transverse direction, an inner sheet (94), an outer sheet (92), and one or a plurality of outer sheet fold overs (931, 932) wherein the outer sheet fold over is an extension of the outer sheet material formed by folding the outer sheet material at the distal edge (88) of the front and back elastic belts or otherwise; wherein the belt elastic members (96F, 96S) are sandwiched between two of these sheets. The front elastic belt (84) and the back elastic belt (86) may each be made only by clastic members (96S), the inner sheet (94), the outer sheet (92), and the outer sheet fold overs (931, 932). At least some of the elastic members (96F, 96S) extend in the transverse direction substantially parallel to each other. All of the elastic members (96F, 96S) may extend in the transverse direction substantially parallel to each other. Such an article may be economically made. At least 10%, or at least from about 15% to not more than about 70%, of the front and back elastic belts (84, 86) from the waist opening in the longitudinal direction may be a laminate in active elasticity along the entire transverse dimension W1 of the front and back elastic belts (84, 86). For each front and back elastic belt (84, 86), the region overlapping the absorbent main body (38) may be removed of its elastic activity. Such region removed of elastic activity is referred herein as the “elastic cut window”, and the remainder of the intact elastic member capable of imparting elasticity is defined as the “effective length of elasticity of an elastic member”.

The tensile stress (N/m) of the entirety of the front and back elastic belts (84, 86), respectively, may be profiled in order to provide the functional benefits of the present invention, such as case of stretch and application, while also maintaining certain force during wear, to prevent the article from sagging after loading. When the elasticity of the front and back elastic belts (84, 86) are provided by a plurality of elastic members (96F, 96S) running in the transverse direction, the tensile stress may be adjusted by one or more of the following methods; 1) elongation rate of the elastic member (96F, 96S); 2) density (dtex) of the elastic member (96F, 96S); 3) longitudinal pitch of multiple elastic members (96F, 96S); and 4) effective length of elasticity of the elastic member (96F, 96S) in the transverse direction. By elongation, “0% elongation” is meant the original length of the elastic member.

Referring to FIG. 3A, the back elastic belt (86) may each be divided into multiple zones spanning in the transverse direction and defined by its location from the distal edge (88) to the proximal edge (90) relative to the percentage of the seam length LS wherein the distal edge (88) is considered 0% and the proximal edge (90) is considered 100%. The multiple zones may be configured to provide different functions to the back elastic belt (86), respectively, a back waist zone (102), a protective zone (103), and a buttock zone (105) lined up from the distal edge (88) to the proximal edge (90), each zone spanning in the transverse direction. In the absorbent article of the present invention, the protective zone (103) comprises a main portion and a waist guard portion (WGP) superposed with each other, the waist guard portion (WGP) extending towards the crotch region from a transversely extending closed end line (103B) towards an open edge, wherein the waist guard portion (WGP) is partially bonded to the main portion to define a pair of closed side lines (103S), wherein a pocket is formed by the closed end line (103B), the closed side lines (103S), and the open edge, the pocket having an area A2 of from about 5% to about 15% or from 10% to about 15% of the back elastic belt area A1; and the closed end line (103B) is positioned proximal from the back distal edge of the backsheet. Elastic members may be disposed on each of the waist zone (102), the protective zone (103) the main portion, and the buttock zone (105).

Referring to FIG. 3A, the closed side lines (103S) may not superpose the backsheet for securing the area of the pocket A2. The pocket may have a transverse dimension W3, wherein W3/W1 is from about 15% to about 60%, or from about 35% to about 50%. When the distance between the side edge (48) of the absorbent main body (38) in the protective zone (103) and the closest transverse edge of the back belt is W4, and a distance between the side edge (48) of the absorbent main body (38) in the protective zone (103) and the closest closed side line is W5, W5/W4 may be from about 5% to about 30%, or from about 10% to about 20%. The pocket may have a longitudinal dimension LP, wherein LP is from about 15% to about 45%, or from about 20% to about 40% of LS.

When the distal edge of the side seam is considered 0% and the proximal edge of the side seam is considered 100%, the protective zone may be disposed within a location of from about 15% to about 80% of LS. The protective zone, namely LP, may not extend the entire longitudinal dimension of from about 15% to about 80% of LS. When the distal edge of the side seam is considered 0% and the proximal edge of the side seam is considered 100%, the protective zone may be disposed within a location of from about 20% to about 75%, or from about 30% to about 75%, or from about 30% to about 65%, or from about 35% to about 65%, of LS. Without being bound by theory, by providing the protective zone in such dimension on the back elastic belt (86), the buttock cleavage of a wearer is effectively covered by the waist guard portion WGP so that low viscosity exudate, such as runny fecal matter, may be effectively drawn into the pocket.

Referring to FIGS. 2A, 3A-3C and 4, the waist guard portion WGP is explained by utilizing an embodiment provided on the back elastic belt (86) of a uni-body type pant. The waist guard portion WGP is made by an extension of the outer sheet (92) extending beyond the longitudinal dimension of the inner sheet (94), which extension of the outer sheet (92) is folded inwardly 2 times in order to encompass a first elastic member (96F) and further to provide the waist guard portion WGP in the intended position relative to the side seam length LS. As a result of the 2 folds, the first clastic member (96F) is sandwiched between the first outer sheet fold (931) and the second outer sheet fold (932), wherein the first outer sheet fold (931) constitutes the garment facing side of the waist guard portion WGP, and the second outer sheet fold (932) constitutes the body-facing surface. Referring to FIGS. 3A-3C, a certain longitudinal dimension from the distal edge of the second outer sheet fold (932) is bonded to the remainder of the belt, such bonding shown as SWFA, however, leaving some longitudinal dimension unbonded. The position provided most distal of the unbonded region is the closed base line (103B), and the common proximal edge of the first outer sheet fold (931) and second outer sheet fold (932) is the open edge (103M). The waist guard portion WGP is defined as having a longitudinal dimension spanning from the closed base line (103B) and the open edge (103M), and having a transverse dimension matching the width of the belt to which it is provided. The left and right transverse edges of the waist guard portion WGP are also bonded to the remainder of the belt, such bonding shown as SWFA to define a pair of side close lines (103S). The unbonded region thus provided is the pocket (103P). Without being bound by theory, by providing the pocket (103P) in such configuration, this prevents low viscosity exudate having entered the pocket (103P) from flowing or penetrating out of the pocket (103P) towards the waist opening.

Referring to FIGS. 3A and 4, the first clastic member (96F) provides the waist guard clastic portion WGEP, wherein elasticity may be imparted by one or more elastic strands, clastic ribbons, mechanically activated nonwovens, or otherwise. The waist guard clastic portion WGEP (1031) is defined as having a longitudinal dimension LE spanning from the open edge (103M) to the distal edge of the first clastic member (96F). When the first clastic member (96F) is a plurality of clastic strands, the distal edge of the waist guard elastic portion WGEP is the position of the clastic strand which is positioned most distal. There may be 2-10 clastic strands, or from 2-5 clastic strands, or 2-8 clastic strands, or 3-4 clastic strands, or 3-8 clastic strands, or 4-6 clastic strands disposed on the waist guard clastic portion. LE is smaller than that of the waist guard portion WGP such that a pocket (103P) having containment capacity may be provided. LE may be no greater than about 40 mm, or no greater than about 35 mm, or no greater than about 30 mm, or no greater than about 25 mm, or from 15 mm to about 40 mm, or from about 10 mm to about 35 mm, or from about 10 mm to about 25 mm. In FIGS. 3A-C, the pocket (103P) has a rectangular shape, however, the pocket (103P) may take other shapes by changing the bonding area SWFA such that the closed base line (103B) and the pair of closed side lines are shaped. For example, the closed base line (103B) may be concave or convex toward the pocket (103B) to match the wearer's anatomy. The longitudinal dimension of the pocket (103P) defined as the shortest distance between the closed base line (103B) to the open edge (103M) may be from about 20 mm to about 45 mm, or from about 24 mm to about 42 mm. The waist guard clastic portion (WGEP) may have a longitudinal dimension LE, wherein LE is from about 5% to about 21% of LS. When the distal edge of the side seam is considered 0% and the proximal edge of the side scam is considered 100%, the waist guard elastic portion (WGEP) may be disposed within a location of from about 40% to about 80%, or from about 40% to about 75% of LS.

Referring to FIGS. 3A and 4, the main portion of the back elastic belt superposing the waist guard elastic portion WGEP is defined a correlated portion CP. Namely, the correlated portion CP has the same longitudinal dimension (1031) as the waist guard clastic portion WGEP. The correlated portion CP may be elasticized or may not be elasticized, wherein the tensile stress of the waist guard elastic portion WGEP is higher than that of the correlated portion CP. The correlated portion CP may be elasticized wherein the tensile stress of the waist guard elastic portion WGEP is from about 125% to about 180% of the tensile stress of the correlated portion CP. Without being bound by theory, such tensile stress difference enables the waist guard clastic portion WGEP to be in close contact with the wearer, while pulling away the open edge (103M) from the correlated portion CP, thus opening the pocket (103P) towards the proximal side. Thus, the pocket (103P) is configured to have good containment capacity. The correlated portion CP may have an elastic cut window such that some portion is elasticized, while the transverse center does not interfere with the opening of the pocket (103P). When the waist guard elastic portion WGEP is elasticized by disposing a plurality of clastic strands (96F), the most proximately positioned clastic strand is disposed from no less than 2 mm to 5 mm away from the open edge (103M). Placement of the elastic strand on the very edge of the open edge (103M) may be avoided in order to prevent the waist guard position WGP causing irritation to the wearer. The waist guard elastic portion WGEP may be disposed with a plurality of clastic strands (96F) with a relatively small pitch between each other. The elastic strands (96F) of the waist guard elastic portion WGEP may be disposed at a pitch of no more than about 12 mm, or no more than about 10 mm, or no more than about 6 mm, or between about 2 mm to about 8 mm, or between about 3 mm to about 9 mm, or between about 2 mm to about 6 mm.

The clastic strands (96F) for providing the waist guard elastic portion WGEP may be provided in color such that the existence and function of the waist guard portion WGP is clearly communicated to the wearer or caregiver. At least 2 of the elastic strands for providing the waist guard clastic portion WGEP may have a color which is visible from the body-facing side of the article. The color may be selected from green, blue, purple, or a combination thereof, in order to be easily recognized, while not being confused with the color of containment. The same or similar color may be used for other clastic strands (96S) disposed on the belt to provide a coordinated appearance. For example, those clastic strands on the front waist zone (102) or the back waist zone (102) may have the same or similar color.

Referring to FIG. 4, the longitudinal edges (50) of the absorbent main body (38) may be positioned and bonded between the waist guard portion WGP and the remainder of the belt. This is to avoid having sharp edges of the absorbent main body (38) on the body-facing surface, and also to avoid leakage from the edges. The absorbent main body (38) comprises an absorbent material region (62) which may be surrounded by an absorbent material non-existing region (61), wherein the absorbent material region (62) having a greater thickness than that of the absorbent material non-existing region (61). The absorbent material region (62) may partially superpose the area of the pocket (103P) to provide at least some absorbent capacity in this region. At least about 3%, or at least about 10% of the area of the pocket (103P) may superpose the absorbent material region (62) toward the proximal edge. Even so, the Combined Thickness of the waist guard clastic portion WGEP, the correlated portion CP, and the portion of the absorbent main body (38) sandwiched therebetween may be kept to no more than about 5 mm, preferably no more than about 4 mm. This is so that the thickness of the remainder of the article does not interfere with the opening of the pocket (103P) towards the proximal side due to the elasticity of the waist guard clastic portion WGEP. When the absorbent main body (38) comprises an acquisition system, the acquisition system may be avoided from superposing the pocket (103P). In order to provide the waist guard portion WGP relative to the absorbent main body (38) in the aforementioned way, the distance between the waist opening to the longitudinal edge of the absorbent main body (38) may be adjusted, namely may be shifted toward the back side or the front side. For example, when the waist guard portion WGP is provided on the back clastic belt (86), the distance between the waist opening to the longitudinal edge of the absorbent main body (38) may be made shorter on the back side than the front side.

Referring to FIG. 3A, the absorbent main body (38) may further comprise a pair of inner cuffs (66) provided along the side edges (48) to provide a barrier along both side edges (48) of the absorbent main body (38). The inner cuffs (66) may be made of material having high fluid impermeability, and elasticized by a plurality of inner cuff elastics (67). The waist guard portion WGP may be configured in a certain way relative to the inner cuffs (66) to provide improved function. The inner cuffs (66) may be in active elasticity at the proximal edge of the waist guard portion (103M). By providing the leg cuffs in active elasticity in such position, the combined elasticity from the leg cuffs and waist guard clastic portion WGEP may help hold the inner cuffs (66) and waist guard portion WGP in close contact with the wearer, and further provide additional containment capacity for the pocket (103P).

Referring to FIG. 3A, the back elastic belt (86) may be divided into 3 zones spanning in the transverse direction and defined by its location from the distal edge (88) to the proximal edge (90) relative to the percentage of the seam length LS wherein; 0% to the distal edge of the waist guard portion WGP is a back waist zone (102), the distal edge to the proximal edge of the waist guard portion WGP is a protective zone (103), and the proximal edge of the waist guard portion WGP to 100% is a buttock zone (105). Elastic members may be disposed on each of the waist zone (102), the protective zone (103) and the buttock zone (105).

Referring to FIG. 4, the elastic cover (40) of the present invention may be made by a relatively simple structure comprising no more than the inner sheet (94), the outer sheet (92), the first outer sheet fold (931), the second outer sheet fold (932), and the first and second elastic members (96F, 96S) encompassed within these layers. Any portion of the elastic cover (40) comprising the waist guard portion WGP may be made by less than 6 layers, or made by 3-4 layers, and in a Combined Thickness of no more than about 5 mm, or no more than about 4 mm. Providing the structure simple and relatively thin is advantageous in maintaining breathability and softness of the belt. Further, the clastic cover (40) may be made by elastic strands commonly used in the art having a density of no more than 940 dtex. Certain zones of the elastic cover (40) may be disposed of elastic bodies having a density of no more than about 500 dtex, thus providing a soft fit. The article of the present invention may be provided with a protective measure for preventing leakage of low viscosity bodily exudates from the waist opening, while maintaining good stretchability for case of application, good fit for preventing sagging, good comfort and softness, and good breathability. The present article may have a Waist Circumference Force according to the Whole Article Force Measurement herein of no more than about 9N, or no more than about 8N.

Referring to FIGS. 3C and 4, the back clastic belt (86) may comprise an attachment free area (AFA) between the second outer sheet fold (932) and the main portion devoid of any attachment, for example, devoid of the second waist fold adhesive (SWFA). The attachment free area (AFA) has a longitudinal dimension of at least 8 mm and extending for the entire transverse dimension. By providing such attachment free area (AFA), this may provide a bulky and soft touch to the body-facing surface.

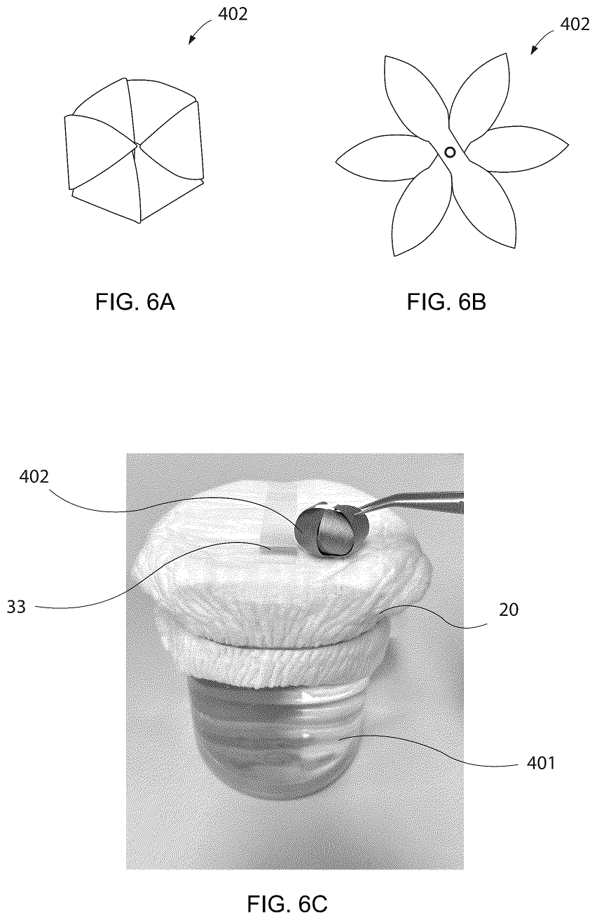

Referring to FIGS. 1, 2A, and 4, even when the back elastic belt (86) comprises a waist guard portion WGP, the remainder of the back clastic belt (86), and particularly the garment facing surface of the back elastic belt (86), may be maintained in a simple structure. This enables the article to have other functions that are desired of a pant type absorbent article, such as having a disposal tape (33) attached to the garment facing surface of the back elastic belt (86), as shown in FIG. 6C. Accordingly, the absorbent article may be devoid of fastening means.

### Demonstration of Vapor Permeability

The absorbent article (20) of the present invention may be subject to a demonstration for visually communicating the thinness and/or controlled number of layers for providing the waist guard portion WGP. Namely, the waist guard elastic portion WGEP may have a Combined Thickness of the waist guard elastic portion, the correlated portion, and the portion of the absorbent main body of no more than about 5 mm, or no more than about 4 mm, and may be made by less than 6 layers, or made by 3-4 layers. Accordingly, the present invention is also directed to a method of demonstrating vapor permeability of the absorbent article in an ambient environment, wherein the method comprises the steps of:
1) providing a vapor supplying source (401) supplying a vapor having a temperature of from about 35° C. to about 95° C., preferably from about 45° C. to about 85° C., in a direction against gravity;
2) placing the wearer facing side of at least a portion of the waist guard elastic portion WGEP in a position capable of receiving the vapor from the vapor supplying source (401);
3) placing a shape memory object (402) having a transformation temperature of at least 30° C. on the garment facing side of the position receiving the vapor, wherein the time on which the shape memory object (402) is placed is considered a start time; and
4) allowing the shape memory object (402) to transform, wherein the transformation starts within about 10 seconds, preferably within about 5 seconds from the start time, and the transformation completes in about 2 to about 30 seconds, preferably in about 3 to about 10 seconds, from the start of transformation.

By ambient environment, what is meant is an environment having a temperature of from about 8° C. to about 25° C., a relative humidity of from about 20% to about 80%, preferably from about 30% to 70%, an air flow of less than about 1.5 m/s, and having a roof or in a closed room.

The vapor supplying source (401) may be any object capable of supplying aqueous vapor and/or hot air flow of at least 40° C., including but not limited to a vessel containing heated water, a humidifier, a hair dryer, a steam iron, or any other object or appliance that is commonly found in a household or a basic research facility.

The shape memory object (402) may comprise any shape memory material which is capable of changing shape at a transformation temperature of at least 30° C., such as a nickel-titanium based shape memory metal alloy (Reference: Hodgson, D. E.; Wu, M. H.; Biermann, R. J. (1990). “Shape Memory Alloys”. Properties and Selection: Nonferrous Alloys and Special-Purpose Materials. pp. 897-902). Materials with either a one-way or two-way shape memory effect may be used, a two-way shape memory effect being preferred. The shape memory object (402) may be designed by assembling the shape memory material components into a certain geometry, or using the shape memory material as a joint or skeleton-like unit to connect with other components made of non-shape memory materials. The shape memory material component may be in the form of sheets, strips wires or coils, and made into a wide range of sizes, thickness, colors, and shapes, and may be combined with non-shape memory components. What is meant by transformation of the shape memory object (402) herein is a change of shape that is easily recognized by the naked eye. Preferably, transformation of the shape memory object (402) is very obviously recognized by the general consumer from a distance of a few meters.

Referring to FIG. 6A, an embodiment of a shape memory object (402) in ambient environment is shown. Specifically, a sheet of nickel-titanium shape-memory alloy material is cut to have the flower petal-like outlines, and a number of the petals are assembled into a flower-like geometry and tightened with the aid of a threaded fastener. The flower-like shaped shape memory object (402) embodiment may have petals in a golden color and a dimension in height and width of from about 2 cm to about 4 cm. The temperature-dependent shape change is directed to a first geometry of a flower bud with curled closed petals at low temperatures as in FIG. 6A and a second geometry of a blooming flower with the petals opened up when reaching about 30° C. or above, as in FIG. 6B. Referring to FIG. 6B, the resulting shape memory object (402) in the second geometry may have a width of from about 4 cm to about 8 cm, enabling easy recognition of transformation.

While not shown, the shape memory object (402) may comprise shape memory material made into joint or skeleton-like units with non-shape memory components attached to such units, for example resembling an animal, or a vehicle, or the like. The first and second geometry may be adjusted to show the shape memory object (402) to have changed posture or have gained mobility.

Referring to FIG. 6C, one embodiment of the demonstration is shown. The vapor supplying source (401) which is a beaker containing heated water is placed with the mouth facing upwards. The wearer facing side of the article (20) including at least a portion of the waist guard elastic portion WGEP is placed on the mouth of the beaker facing the water surface. The article (20) may be attached firmly against the outside walls of the beaker by a rubber band, or the like, in order to receive the aqueous vapor effectively. To the garment facing side of the article thus positioned, the shape memory object (402) is placed, and allowed to transform. A tweezer may be used for placing the shape memory object (402) in the desired position, in order to avoid premature transfer of body temperature of the operator to the shape memory object (402). The position of placement may avoid an optional disposal tape (33) provided on the back belt of the pant article (20), in that such disposal tape (33) may obstruct vapor permeability.

In an alternative embodiment of demonstration, the front and back longitudinal ends of the article may be clamped such that the wearer facing side of the article (20) including at least a portion of the waist guard elastic portion WGEP is facing down, a hair dryer is blown against the wearer facing side of the article (20) from under the article, and the shape memory object (402) is placed on the garment facing side of the portion receiving the hot air vapor to be allowed to transform. Start of blowing the hair dryer and placing the shape memory object (402) may be coordinated to happen at the same time.

The temperature and type of the vapor supplying source (401), the temperature and shape of the shape memory object (402), and the distance between the vapor supplying source and the article may be adjusted, such that the transformation of the shape memory object (402) is provided in a quick enough duration of time that is well recognized by the observer. Further, the article (20) and the vapor supplying source (401) are positioned such that the materials of the article (20) are not distorted by the heat from the vapor supplying source (401). For providing good recognition to the observer, when the time on which the shape memory object (402) is placed is considered a start time; the transformation starts within about 10 seconds, preferably within about 5 seconds from the start time, and the transformation completes in about 2 to about 30 seconds, preferably in about 3 to about 10 seconds, from the start of transformation.

### Linear Dimension Measurements and Area Calculations

To obtain a sample for this measurement, the side seams (32) of an article are opened, and the garment-facing side of the entire article is attached on surface of a non-transparent backboard by hook material, or double side tape material. The backboard should be large enough to support the entire area of the article. When attaching, any elastics are stretched to its “maximum stretched state” wherein the following conditions a)-c) are met, unless any material failure is observed.
a) The side seams are straight.
b) There are no wrinkles on the central chassis or the belt elastic regions.
c) The left part and right part of the article in view of the longitudinal axis are symmetrical.

For most articles, the front and back elastic belt regions may be stretched up to the “30N Circumference” as measured under the “Whole Article Force Measurement” below, for meeting the conditions above. In case any material failure is observed prior to reaching a level of stretch wherein the above conditions are met, then the point of maximum stretch at which the material is yet to fail is considered the “maximum stretched state”. Material failure may include decomposing of nonwoven material, breaking of elastic strands, or the like.

What is measured is the following dimensions of the sample, wherein some of the dimensions are measured differently depending on the contour of the article. FIGS. 2A, 2B, and 3A are representative article contours for explaining how the dimensions are measured, and not intended to be an exhaustive list of article contours of the present invention. The linear dimensions are reported to the nearest 1 mm. The area calculations are reported to the nearest 1 mm². Five samples are measured and their average are obtained.
W1: transverse dimension of the back elastic belt
W2: maximum transverse dimension of the absorbent main body
W3: transverse dimension of the pocket
W4: distance between the side edge of the absorbent main body in the protective zone and the closest transverse edge of the back belt
W5: distance between the side edge of the absorbent main body in the protective zone and the closest closed side line
W6: minimum transverse dimension of the elastic cover
LS: longitudinal dimension of the side seam
LP: longitudinal dimension of the pocket
LE: longitudinal dimension of the waist guard elastic portion
A1: W1×LS
A2: W3×LP

### Combined Thickness

Combined Thickness is measured under 0.1 psi using a caliper measurement device such as PEACOCK 307 from Ono Sokki Inc or the like. A sample for this measurement is prepared in the same manner as in the measurement for “Linear Dimension Measurements and Area Calculations” above.

The approximate center of the waist guard elastic portion in both the longitudinal and transverse direction is identified. The thickness of the article of such approximate center is measured including at least the waist guard elastic portion, the correlated portion, and the portion of the absorbent main body sandwiched therebetween. The dimensions are reported to the nearest 0.01 mm. Five samples are measured and their average is obtained.

### Whole Article Force Measurement

Force is measured using an Electronic Tensile Tester with a computer interface such as the MTS Criterion C42 running TestWorks 4 Software (available from MTS SYSTEMS (CHINA) CO., LTD) or equivalent instrument. A load cell is selected so that force results for the samples tested will be between 10 and 90% of capacity of the load cell used. The instrument is calibrated according to the manufacturer's instructions. All testing is performed in a room maintained at 23±2° C. and 50±5% relative humidity.

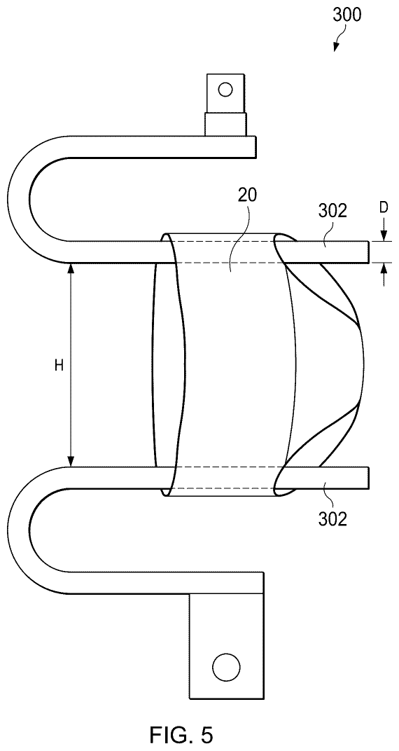

The tensile tester is fitted with hanger-type sample holding fixtures (300) as shown in FIG. 5. Each fixture comprises a rigid linear rubber-coated horizontal bar section (302) to prevent sample slippage during testing. The outer bar diameter (including the rubber coating) of the horizontal bar sections is 10.0 mm. The central axes of the horizontal bar sections (302) are configured to remain parallel and in the same vertical plane throughout the test procedure. The gauge circumference is determined by the following equation:

$$Gauge Circumference = 2 \times ( H + D + π D / 2 )$$

where H is the vertical gap between the horizontal bar sections (302), and D is the outer diameter of the bar.

The instrument is set up to go through the following steps:

| | |
|---|---|
| Crosshead Speed | 254.0 mm/min |
| Final Load Point | 19.61N |
| Hold Time | 0 |
| Number of Cycles | 1 |
| Data Acquisition Rate | 50 Hz |

An article (20) sample is inserted onto the upper horizontal bar section (302) so that the bar passes through the waist opening and one leg opening of the article. The crosshead is raised until the specimen hangs above the lower bar and does not touch lower bar (302). The load cell is tared and the crosshead is lowered to enable the lower bar (302) to be inserted through the waist opening and other leg opening without stretching the article. The article is adjusted so that the longitudinal centerline L1 of the article is in a horizontal plane halfway between the upper and lower bars (302). The center of the side portion in contact with the bar (302) is situated on the same vertical axis as the instrument load cell. The crosshead is raised slowly while the article is held in place by hand as necessary until the force is between 0.05 and 0.1N, while taking care not to add any unnecessary force. The gauge circumference at this point is the Initial Gauge Circumference. The test is initiated and the crosshead moves up at 254 mm/min until a force of 19.6N is attained, then the crosshead immediately returns to the Initial Gauge Circumference at the same speed. The maximum circumference at 19.6N and the force at 70% of the maximum circumference during the loading segment and unloading segment of the test are recorded.

The maximum circumference at 19.6N is defined as the Full Stretch Circumference (mm). The Full Stretch Circumference (mm)×0.7 is defined as the 70% Stretch Circumference. The Waist Circumference Force is defined as the force at 70% stretch circumference during the load (extension) segment of the test. Five samples are analyzed and their average are calculated and reported to the nearest 0.01N, respectively.

### Belt Zone Tensile Stress Measurement

The tensile stress (N/m) is calculated by tensile force (N) divided by the specimen width (m). Force may be measured using an Electronic Tensile Tester with a computer interface such as the MTS Criterion C42 running TestWorks 4 Software (available from MTS SYSTEMS (CHINA) CO., LTD) or equivalent instrument. A load cell is chosen so that force results for the samples tested will be between 10% and 90% of capacity of the load cell. The instrument is calibrated according to the manufacturer's instructions. All testing is performed in a room maintained at 23±2° C. and 50±5% relative humidity. The instrument is equipped with single line contact grips at least as wide as the test specimen.

To obtain test specimens, the sample article is cut open along the side seams (32), and the elastic cover (40) or the front and belt regions (26, 28) are removed from the absorbent main body (38) by separating the bonding between them, and further the waist guard portion WGP is separated from the belt to which it is attached. Cold Spray may be used, paying attention not to make wrinkles in the belt sections. Care is taken not to spray on any belt elastic member (96F, 96S). The obtained elastic belt samples are severed into zones (102, 103, 1031-WGEP, 1031-CP, 105) according to the present invention with care not to cut any elastic member (96F, 96S). Samples are pre-conditioned at 23° C.±2 C.° and 50%±5% relative humidity for two hours prior to testing.

The instrument is set up to go through the following steps. Initial Gauge Length is calculated from the Initial Gauge Circumference which is determined during the Whole Article Force Test using separate identical articles, as described above. Initial Gauge Length=0.5×Initial Gauge Circumference. The final gauge length is calculated from the Full Stretch Circumference which is determined during the Whole Article Force Test, as described above.

| | | |
|---|---|---|
| Crosshead Speed | 254.0 | mm/min |
| Data Acquisition Rate | 50 | Hz |

| | |
|---|---|
| Final Gauge Length | 0.5 × Full Stretch Circumference |
| Hold Time | 0 |
| Number of Cycles | 1 |

One end of the specimen is clamped into the upper clamp and the load is tared. The other end of the specimen is clamped into the lower clamp. Approximately 5 mm of each end of the specimen is behind the contact line of the grip. The test is started and the specimen is extended to the final gauge length at a crosshead speed of 254 mm/min, then immediately returned to the original gauge length at the same speed. The specimen is extended in the article transverse direction during the test. The unload force at 70% of the Final Gauge Length during the unload segments of the test is recorded.

Five articles are analyzed and the unload forces are recorded for each of the zones. The average tensile force (N) is calculated to the nearest 0.01 N for each. The tensile stress for each zone is calculated by the average tensile force (N) divided by the average specimen width (m) and reported to the nearest 0.1 N/m.

### EXAMPLES

### Example 1

One piece-type pant absorbent article of the present invention in Size 4 (L size) having an elastic profiling of the back elastic belt according to Table 1 below. The protective zone comprising the waist guard portion WGP was positioned at from 39% to 65% of LS on the back elastic belt. The waist guard elastic portion WGEP was positioned at from 52% to 65% of LS on the back elastic belt and had 3 elastic strands. The 2 elastic strands on the correlated portion CP had an elastic cut window.

### Comparative Example 1

One piece-type pant absorbent article in Size 4 (L size) sold by the tradename of “Pampers Luxurious Skin” manufactured for the Chinese market however having the core replaced with Lot No. FJTB2K257B01 the core manufactured by Fujian Tengbang New Material Co., Ltd. The replaced core is different from that of “Pampers Luxurious Skin” only in the pore size of nonwoven material. The replaced core had the same dimension of parts and same absorbency capacity of superabsorbent polymers as “Pampers Luxurious Skin”. The front elastic belt and absorbent main body had a very similar size, structure, and elastic profiling as Example 1. The back elastic belt size is the same size as Example 1 while being devoid of a waist guard portion WGP and having an elastic profiling of the back elastic belt according to Table 1 below in corresponding positions.

TABLE 1

| | dtex / elongation % / number of elastic members | |
|---|---|---|
| | (dimension of elastic cut window) | |
| | Example 1 | Comparative Example 1 |
| Waist zone | 620 dtex / 200% / 5 | 620 dtex / 200% / 7 |
| | 940 dtex / 200% / 2 | |
| Protective zone Waist Guard | 620 dtex / 180% / 3 | N/A |
| Portion | | |
| Protective zone Main portion | 470 dtex / 140% / 2 (110 mm) | 620 dtex / 170% / 2 |
| Buttock zone | 470 dtex / 140% / 2 (110 mm) | 620 dtex / 170% / 4 (110 mm) |
| | 620 dtex / 170% / 2 (110 mm) | |
| A2 (mm²) / A1 (mm²) = (%) | 5252/44850 = 12% | N/A |
| W2 (mm) / W1 (mm) = (%) | 136/390 = 35% | 136/390 = 35% |
| W3 (mm) / W1 (mm) = (%) | 175/390 = 45% | N/A |
| W5 (mm) / W4 (mm) = (%) | 20/127 = 16% | N/A |
| W6 (mm) | 161 | 161 |

For Examples 1 and Comparative Example 1, the Waist Circumference Force according to the Whole Article Force Measurement herein, and the tensile stress of the waist guard elastic portion WGEP and the correlated portion CP according to the Belt Zone Tensile Stress Measurement herein, were measured. Results are provided in Table 2 below.

TABLE 2

| | Example 1 | Comparative Example 1 |
|---|---|---|
| Waist Circumference Force (N) | 3.66 | 3.03 |
| Tensile stress of waist guard elastic portion (N/m) | 15.0 | N/A |
| Tensile stress of correlated portion (N/m) | 10.0 | N/A |
| Combined Thickness (mm) | 2.69 | N/A |

### Consumer Acceptance

80 panelists who were caregivers of babies using Size 4 (L size) weighing 9-14 kg and 0-36 months in age, with approximately equal number of males and females, and having a mixture of usage experience of major brands of similar price range, were recruited. Upon recruiting, each of the panelists were asked to confirm if their baby had experience of low viscosity fecal matter leakage. Each panelist was asked to use each test samples at various sequence for 5 days each, and enough samples were provided to test each test sample. The panelists were asked to report the number of samples which were contained with urine/fecal matter, and the number of incidents of leakage of urine/fecal matter from the back waist. The panelists were also asked to rate the test samples against those values using 5 ratings (Overall rating), which were scored as such: “Poor”=0, “Fair”=25, “Good”=50, “Very Good”=75, and “Excellent”=100. The panelists were also asked to rate the test samples against those values using 5 ratings (Relative Category Rating), which were scored as such: “The best diaper I have used”=100, “Better than the diaper I have used”=75, “No difference vs. the diaper I have used”=50, “Worse than the diaper I have used”=25, “The worst diaper I have used”=0. The result of the ratings and percentages of leakage are provided in Table 3.

TABLE 3

| Value | Example 1 | Comparative Example 1 |
|---|---|---|
| Overall rating | 69 | 71 |
| Relative Category Rating | 60 | 64 |
| Urine leakage % | 0.9% | 0.7% |
| Fecal matter leakage % | 4.0% | 1.8% * |

* statistically significant at 90% confidence level

Example 1 as well as Comparative Example 1 provide good fit, comfort during wear, urine leakage prevention, and softness. Compared to Comparative Example 1, Example 1 provides good protection against leakage of fecal matter from the waist opening on the back side at a statistically significant level. Example 1 may be manufactured at substantially the same speed as manufacturing Comparative Example 1, and at an upcharge of material cost of no more than about 5%.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article having a longitudinal direction, a transverse direction, a pair of side seams, and a pair of leg openings, comprising:
a front elastic belt transversely extending between the pair of side seams;
a back elastic belt transversely extending between the pair of side seams;
a crotch region longitudinally extending between the front and back elastic belts; and
an absorbent main body extending the entire longitudinal dimension of the crotch region and further extending partly into each of the front elastic belt and the back elastic belt, the absorbent main body comprising a liquid pervious topsheet, a liquid impervious backsheet, and an absorbent material region sandwiched therebetween, the absorbent main body having a maximum transverse dimension W2 which is from about 22% to about 50% of a transverse dimension of the back elastic belt W1;
the back elastic belt divided into a back waist zone, a protective zone, and a buttock zone lined up from the distal edge to the proximal edge, each zone spanning in the transverse direction and defined by its location from the distal edge to the proximal edge relative to the percentage of a longitudinal dimension LS of the side seam, wherein elastic members are disposed on each of the back waist zone, the protective zone, and the buttock zone;
the protective zone comprising a main portion and a waist guard portion superposed with each other, the waist guard portion extending towards the crotch region from a transversely extending closed end line towards an open edge, wherein the waist guard portion is partially bonded to the main portion to define a pair of closed side lines, wherein a pocket is formed by the closed end line, the pair of closed side lines, and the open edge, the pocket having an area A2 of from about 5% to about 15% of the back elastic belt having an area A1; and the closed end line is positioned proximal from the back distal edge of the backsheet; and
wherein the waist guard portion comprises a waist guard elastic portion, wherein the main portion superposing the waist guard elastic portion is defined a correlated portion.

2. The absorbent article of claim 1, wherein the closed side lines do not superpose the backsheet.

3. The absorbent article of claim 2, wherein the pocket has a transverse dimension W3, wherein W3/W1 is from about 15% to about 60%.

4. The absorbent article of claim 2, further comprising:
a distance between the side edge of the absorbent main body in the protective zone and the closest transverse edge of the back belt W4, and
a distance between the side edge of the absorbent main body in the protective zone and the closest closed side line W5, and
wherein W5/W4 is from about 5% to about 30%.

5. The absorbent article of claim 1, wherein when the distal edge of the side seam is considered 0% and the proximal edge of the side seam is considered 100%, the protective zone is disposed within a location of from about 15% to about 80% of LS.

6. The absorbent article of claim 1, wherein the pocket has a longitudinal dimension LP, wherein LP is from about 15% to about 45% of LS.

7. The absorbent article of claim 1, wherein the back elastic belt is formed by an inner sheet, an outer sheet, a first outer sheet fold over, a second outer sheet fold over, a first plurality of elastic strands, and a second plurality of elastic strands;
wherein a longitudinal dimension of the second outer sheet fold over is defined by a first waist fold point and a second waist fold point, wherein the first plurality of elastic strands are sandwiched between the first outer sheet fold over and second outer sheet fold over, and the second plurality of elastic strands are sandwiched between the inner sheet and the outer sheet; and
wherein the first outer sheet fold over, the second outer sheet fold over, and the first elastic strands form the waist guard elastic portion.

8. An absorbent article having a longitudinal direction, a transverse direction, a pair of side seams having a longitudinal dimension LS, and a pair of leg openings, comprising:
a front belt transversely extending between the pair of side seams;
a back belt transversely extending between the pair of side seams;
a crotch region longitudinally extending between the front and back elastic belts; and
an absorbent main body extending the entire longitudinal dimension of the crotch region and further extending partly into each of the front elastic belt and the back elastic belt, the absorbent main body comprising a liquid pervious topsheet, a liquid impervious backsheet, and an absorbent material region sandwiched therebetween, the absorbent main body having a maximum transverse dimension W2 which is from about 22% to about 50% of a transverse dimension of the back elastic belt W1;
the back elastic belt formed by an inner sheet, an outer sheet, a first outer sheet fold over, a second outer sheet fold over, a first plurality of elastic strands, and a second plurality of elastic strands,
wherein a longitudinal dimension of the second outer sheet fold over defined by a first waist fold point and a second waist fold point, wherein the first plurality of elastic strands are sandwiched between the first outer sheet fold over and second outer sheet fold over, and the second plurality of elastic strands are sandwiched between the inner sheet and the outer sheet;
the first outer sheet fold over, the second outer sheet fold over, and the first elastic strands forming a waist guard elastic portion wherein the waist guard elastic portion has a longitudinal dimension defined by the distance between the first waist fold point and the most distal positioned first elastic strand, wherein the waist guard elastic portion is partially bonded to the remainder of the back belt to define a pair of closed side lines and a nonbonded region between the closed side lines; and
the distance between the closed side lines having a transverse dimension W3, wherein W3/W1 is from about 15% to about 60%, and wherein the closed side lines do not superpose the backsheet.

9. The absorbent article of claim 8, wherein when the distal edge of the side seam is considered 0% and the proximal edge of the side seam is considered 100%, the waist guard elastic portion is disposed within a location of from about 40% to about 80% of LS.

10. The absorbent article of claim 8, wherein the waist guard elastic portion has a longitudinal dimension LE, wherein LE is from about 5% to about 21% of LS.

11. The absorbent article of claim 8, further comprising an elastic cover extending the entire longitudinal dimension of the article and comprising the front elastic belt and the back elastic belt, the absorbent main body having a maximum transverse dimension W2, wherein the elastic cover having a minimum transverse dimension W6 which is from about 110% to about 150% of W2.

12. The absorbent article of claim 8, wherein the back elastic belt comprises an attachment free area between the second outer sheet fold and the main portion, wherein the attachment free area is devoid of any attachment, the attachment free area having a longitudinal dimension of at least 8 mm and extending for the entire transverse dimension.

13. The absorbent article of claim 8, wherein the absorbent main body comprises a pair of inner cuffs wherein the inner cuffs are in active elasticity at the point intersecting the proximal edge of the waist guard portion.

14. The absorbent article of claim 8, wherein the tensile stress of the waist guard elastic portion is from about 125% to about 180% of the tensile stress of the correlated portion.

15. The absorbent article of claim 8, wherein the waist guard elastic portion is elasticized by disposing a plurality of elastic strands, wherein the most proximately positioned elastic strand is disposed from no less than about 2 mm to about 5 mm away from the open edge.

16. The absorbent article of claim 15, wherein the plurality of elastic strands of the waist guard elastic portion are disposed at a pitch of no more than about 12 mm, preferably no more than about 10 mm.

17. The absorbent article of claim 15, wherein at least 2 of the elastic strands of the waist guard elastic portion have a color which is visible from the body-facing side of the article.

18. The absorbent article of claim 8, wherein the Combined Thickness of the waist guard elastic portion, the correlated portion, and the portion of the absorbent main body sandwiched therebetween is no more than about 5 mm.

19. The absorbent article of claim 8, wherein the Waist Circumference Force according to the Whole Article Force Measurement herein is no more than about 9N, preferably no more than about 8N.

20. A method of demonstrating vapor permeability of the absorbent article of claim 18 in an ambient environment, wherein the method comprises:
providing a vapor supplying source supplying a vapor having a temperature of from about 35° C. to about 95° C., preferably from about 45° C. to about 85° C., in a direction against gravity;
placing the wearer facing side of at least a portion of the waist guard elastic portion in a position capable of receiving the vapor from the vapor supplying source;
placing a shape memory object having a transformation temperature of at least 30° C. on the garment facing side of the position receiving the vapor, wherein the time on which the shape memory object is placed is considered a start time; and
allowing the shape memory object to transform, wherein the transformation starts within about 10 seconds, preferably within about 5 seconds from the start time, and the transformation completes in about 2 to about 30 seconds, preferably in about 3 to about 10 seconds, from the start of transformation.
